(19) United States
(12) Patent Application Publication
(10) Pub. No.: US 2022/0114675 A1
(43) Pub. Date: Apr. 14, 2022

(54) AUDIT TRAIL AND AUTO REIMBURSEMENT

(71) Applicant: Healthcare Integrated Technologies Inc., Knoxville, TN (US)

(72) Inventors: Jurgen VOLLRATH, Sherwood, OR (US); Kenneth M. GREENWOOD, Davenport, FL (US); Scott Michael BORUFF, Knoxville, TN (US)

(21) Appl. No.: 17/500,852

(22) Filed: Oct. 13, 2021

Related U.S. Application Data
(60) Provisional application No. 63/204,623, filed on Oct. 14, 2020.

(51) Int. Cl. G06Q 40/08; G16H 10/60; G06Q 50/26; G16H 40/20

(52) U.S. Cl. CPC G06Q 40/08; G16H 40/20; G06Q 50/265; G16H 10/60

(57) ABSTRACT

In an automatic reimbursement and audit system, medical activities are broken into tasks that correspond to tasks required for compliance with CPT codes, users are guided through the required tasks, data associated with the tasks is captured and stored, and payers are contacted for reimbursement once all of the tasks required for a CPT code reimbursement have been complied with.

### FIELD OF THE INVENTION

The invention relates to medical records and reimbursement.

### BACKGROUND OF THE INVENTION

A large percentage of a physician and medical facilities overhead is dedicated to maintaining medical records and correctly billing insurance companies and patients for services rendered. Some electronic medical records address the issue of record keeping and help in identifying the services performed to verify compliance with billing codes (also referred to herein as Current Procedural Terminology, or CPT codes). But these typically require extensive additional work in billing for the work performed and seeking reimbursement of payers.

### SUMMARY OF THE INVENTION

The present invention provides for a system for capturing the tasks performed as part of a medical activity, e.g. a diagnosis, surgery, or other service provided for a patient. By comparing the tasks to those required for compliance with one or more CPT codes, or by presenting a user with the tasks required for a CPT code, the present system supports the user to ensure that all of the tasks for a particular code are performed. Insofar as one or more tasks are missing from completion of a reimbursable medical activity (CPT code activity) the system may prompt the user to perform the missing tasks. Once all of the tasks for completion of a CPT code activity have been completed, the system may automatically bill the payer (e.g. Medicaid, Medicare, Insurance Provider, patient, etc) based on medical insurance information made available to the user.

The system may be integrated with a user's Electronic Medical Record (EMR) system and may include a database of CPT codes, each associated with a list of tasks that need to be performed in order to comply with reimbursement requirements. In such an embodiment the patient's insurance information may already have been entered into the EMR system. The system of the invention may instead be implemented as a separate data capture platform, e.g. native mobile app that is installed on a user's smart phone, or a web app that works via a web browser e.g. tablet or smartphone, and requires either a cell signal or wi-fi to function. A user in such a situation may again be presented with a data capture page for entering details of tasks to be performed or checking off completion of requisite tasks associated with a medical activity, and associating these with necessary tasks for compliance with one or more CPT codes. The user can again be prompted for completion of tasks or presented with additional tasks to be performed in order to complete a CPT code activity.

According to the invention there is provided a system for billing for medical services, comprising a database or other data memory configuration for storing data, the data including CPT codes and associated activities required for reimbursement under each CPT code; a processor; a communications system for communicating with client devices and payers, and control memory in communication with the processor, wherein the control memory includes machine readable code for controlling the processor and the communications system. The system may be implemented as a server in communication with one or more client devices that capture data relating to tasks performed by one or more user clinicians on the client devices.

At least some of the client devices may include a memory configured with machine readable code defining an electronic medical record (EMR).

The server may comprise a dispersed server system connected by internet connections with the client devices, e.g., a cloud server or edge computing configuration.

The machine-readable code may include one or more algorithms for comparing data received from client devices relating to tasks performed against those required for reimbursement under one or more CPT codes, and requesting reimbursement from one or more payers when tasks by a user clinician on a client device correspond to tasks required for reimbursement under one or more CPT codes.

The data received from client devices typically includes patient insurance information to identify the one or more payers.

The request for reimbursement or communications with clients may be based on cell phone or on-line communications.

Further, according to the invention, there is provided a method of billing for a medical service, comprising: capturing on a client device, the steps taken by a user clinician in performing the medical service, comparing the steps to predefined steps associated with one or more reimbursable procedures as defined by CPT codes, identifying reimbursement compliance with all of the predefined steps associated with one or more CPT codes, and automatically requesting reimbursement from one or more payers based on the said compliance identification.

The method may include notifying the clinician user of one or more missing tasks required for reimbursement compliance with one or more CPT codes.

The method may further include capturing patient insurance information for a patient for whom a medical activity or service is performed.

The capturing of one or more of: the tasks performed by a user clinician in performing the medical service or activity, and patient insurance information, may include extracting the data from the electronic medical record (EMR) for the patient for whom the medical activity is performed.

### BRIEF DESCRIPTION OF THE DRAWINGS

FIG. 1 shows one embodiment of a system of the invention;

FIG. 2 shows one embodiment of a user interface for capturing tasks, and

FIG. 3 is a flow chart of one implementation of the present invention.

### DETAILED DESCRIPTION OF THE INVENTION

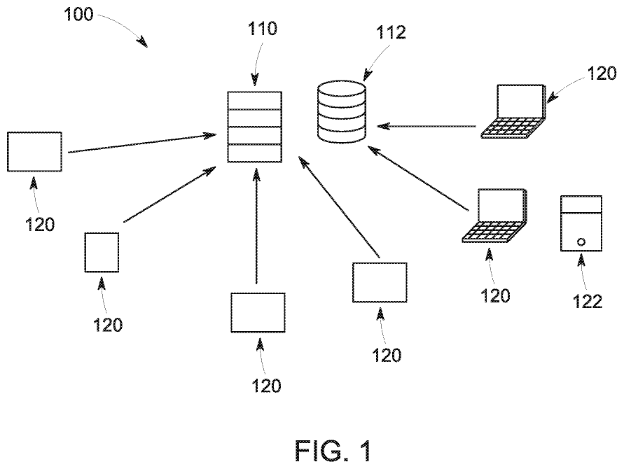

One embodiment of a system of the invention is shown in FIG. 1. The system 100 includes a processor, which in this embodiment comprises a server 110. The server includes a memory for data storage, which in this embodiment comprises a database 112.

In accordance with the invention, the system 100 is connected to client devices 120, which may take the form of desktop computers 122, smart phones, tablets or laptops. The client devices in this embodiment include means for accessing a user interface platform to facilitate data entry and communication with the server 110.

The system includes a communications system, which in this embodiment comprises a Wifi transceiver that allows the server 110 to communicate with the clients 120. The user interface platform may include either a native mobile application (App) that is downloaded onto the client device, or comprises a web app on the server that is accessible from a client device 120 using a browser and HTTP. The user interface platform may also be integrated into a clinician's EMR (Electronic Medical Record) system. Thus, the desktop 122 may already include an EMR system that captures patient information and tasks performed by a physician or other medical staff. In such an implementation the user interface platform of the present invention may supplement the EMR user interface to provide the medical practitioner with feedback on what tasks have been performed, what tasks may be missing for compliance with a particular CPT code, and prompts for additional tasks that may be performed in order to complete a reimbursable CPT code activity.

Thus, according to one aspect of the invention, there is provided a method for assisting medical practitioners and other persons performing remote medically reimbursable tasks (e.g. installation of patient sensors and telemedicine platforms and teaching patients to use such sensors and platforms). In particular, the present invention assists such providers with a user platform for recording or otherwise capturing the tasks they perform and relating the tasks to the necessary tasks required for compliance with one or more CPT codes. The system of the present invention is configured by machine-readable code on a memory connected to the processor (server 110 in FIG. 1) to compare tasks performed by a provider (also referred to herein as a user clinician) to pre-defined tasks associated with CPT codes. The CPT codes and pre-defined tasks are maintained in the database 112. Once all of the tasks corresponding to a particular CPT code have been performed, the server connects with one or more payers (e.g. Medicare, Medicaid, insurance company, or patient) based on patient medical insurance data that is provided by the client device 112.

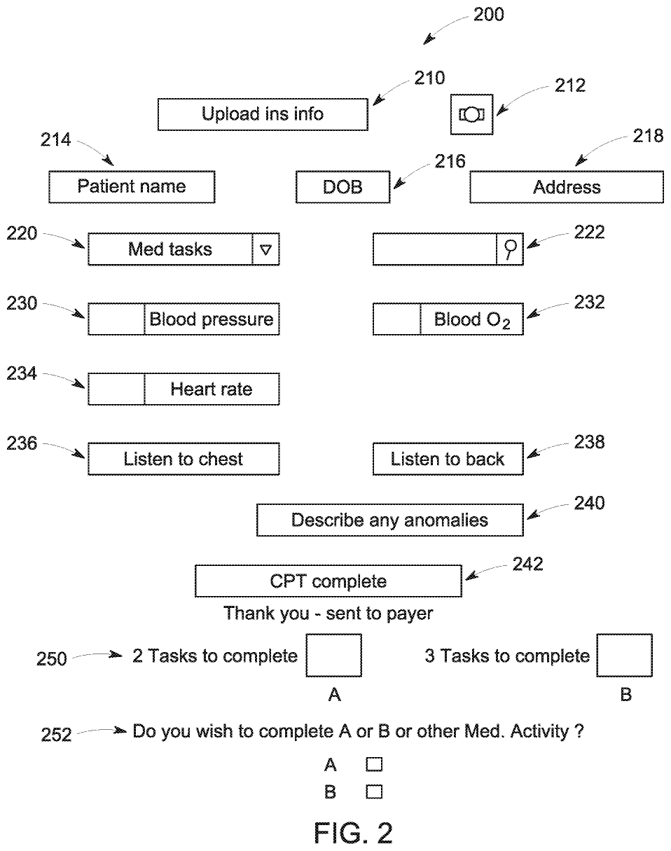

A medical provider's activity may commence with the provider (user clinician) or support staff uploading patient insurance information (upload field 210) or taking a picture of the patient's insurance card (field 212) using the user interface discussed above (one embodiment of which is shown in FIG. 2). Patient verification is provided by entering or uploading the patient's name (field 214), date of birth (field 216) and Address (fields 218).

The data capture associated with the medical activity may include selecting the particular activity that is to be performed from a drop-down menu 220. The user may instead search for related activities using a search field 222.

The system identifies one or more CPT codes and associated activities for the user clinician to choose from (not shown) and, based on the choice, presents the user with a set of fields that require data entries or compliance checks by the user clinician. In this case the fields 230-240 have been identified as part of a general check-up, including blood pressure (field 230), blood oxygenation (field 232), heart rate (field 234), stethoscope examination of chest (field 236) and back (field 238) and comment field 240 for recording any anomalies detected. Once all of the fields have had their data entered or have been clicked to indicate compliance with the defined step, the system submits a confirmation message 242 to the user interface indicating that all tasks have been performed for the identified CPT code and that a request for reimbursement has been submitted to the necessary insurance entities (payers). In another embodiment, the data associated with the activities performed by a clinician are first uploaded into the patient's electronic medical record (EMR) whereafter the request for reimbursement is sent out to the payers from the EMR system. Since the system of the present invention may be in communication with the EMR used by the clinician, it may capture insurance (payer) information from the EMR and may request reimbursement from the payer directly. The system of the invention may also capture data of one or more of the activities performed by the clinician, from the patient's EMR instead of having the clinician entering the data directly into the system via a client device.

In this embodiment the user clinician is prompted with the option of performing additional tasks by identifying reimbursable activities that require only 1, 2, or 3 additional tasks (prompt 250).

The user is presented with selection boxes 252 to select one of the proposed additional activities (which will then present the user with the additional tasks to be performed) or to select any other activity or select DONE to terminate the session for that client.

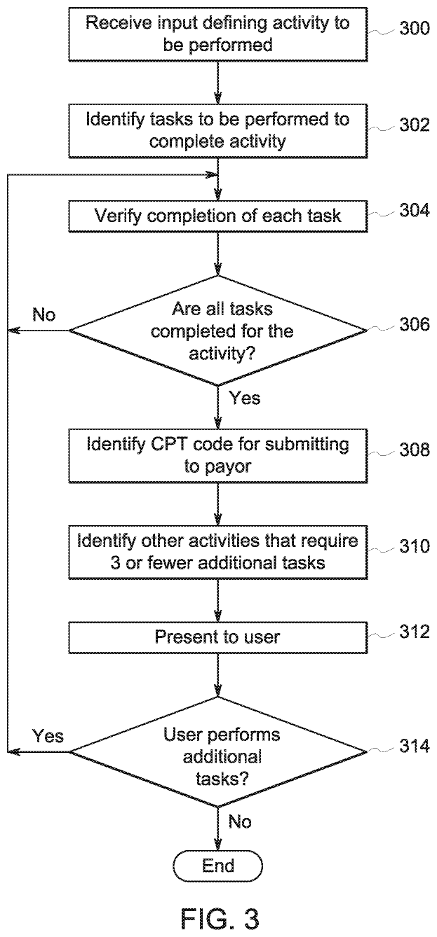

One embodiment of the logic of the machine-readable code on the memory that is connected to the processor (such as server 110 in FIG. 1) is shown in FIG. 3. In step 300 the activity to be performed on a patient captured by the system, e.g., by entering the information using one of the client devices. The tasks to be performed in order to constitute a reimbursable activity, are identified (Step 302). The logic then loops through each task in an activity by checking on completion of the task (Step 304) and then determining whether there are any remaining tasks in the activity (Step 306). In step 308 the corresponding CPT code is identified for submission to payor for reimbursement. The logic in this embodiment also identifies other reimbursable activities that require only a few additional tasks to be performed (in this case 3 or fewer tasks) (Step 310) and presents any such activities and associated tasks to the user (Step 312). In step 314, if the user decides not to perform any additional tasks the logic flow ends and the logic is completed when the request for reimbursement and CPT code were submitted to the payor in Step 308. Otherwise, the logic loops back to the step 304 to verify completion of the additional tasks.

According to one aspect of the present invention, records are kept of all activities performed for a client, which may be fed back into an existing electronic medical record (EMR) of the patient or may be used to upload into a new EMR once a user clinician implements an EMR system. Keeping a record for each patient also serves as a compliance record in the event of future audits or law suits, to provide a record trail of what activities were performed and what tasks were completed in compliance with such activities.

While the present invention was described with respect to particular embodiments, it will be appreciated that the invention could be implemented in different ways, using different configurations and workflows without departing from the scope of the invention. The hardware implementation may also vary, e.g., the server 110 may comprise a dispersed server system (cloud server) like Amazon Web Services (AWS), and communications between clients and server or between server and payers may include cell phone connections. The processing can also be implemented using a processor on the user device rather than involving a client-server arrangement as discussed above.

What is claimed is:

1. A system for billing for medical services, comprising
a database or other data memory configuration for storing data, the data including CPT codes and associated activities required for reimbursement under each CPT code;
a processor;
one or more client devices for entering data,
a communications system for communications between the processor and said client devices and with payers, and
control memory in communication with the processor, wherein the control memory includes machine readable code for controlling the processor and the communications system.

2. A system of claim 1, wherein the processor is defined by a server in communication with said client devices.

3. A system of claim 2, wherein at least one of the client devices includes a client processor and a client memory connected to the client processor, configured with machine readable code for accessing or defining an electronic medical record (EMR).

4. A system of claim 3, wherein the client memory of said one or more client devices is configured with a native mobile app or a browser for accessing a web app.

5. A system of claim 2, wherein the server comprises a dispersed server system connected with the client devices.

6. A system of claim 1, wherein the machine-readable code on the control memory includes one or more algorithms for comparing data received from client devices to activities required for reimbursement under one or more CPT codes, and requesting reimbursement from one or more payers when data from one or more of the client devices corresponds to activities for reimbursement under one or more CPT codes.

7. A system of claim 6, wherein the data received from client devices or data on the data memory includes patient insurance information to identify the one or more payers.

8. A system of claim 7, wherein the request for reimbursement or communicating with client devices is based on cell phone communications.

9. A method of billing for a medical service, comprising
capturing on a client device, the steps taken by a clinician in performing the medical service,
comparing the steps to predefined steps associated with one or more reimbursable procedures as defined by CPT codes,
identifying reimbursement compliance with all of the predefined steps associated with one or more CPT codes, and
requesting reimbursement from one or more payers based on said reimbursement compliance identification.

10. A method of claim 9, further comprising notifying the clinician of one or more missing steps required for reimbursement compliance with one or more CPT codes.

11. A method of claim 9, further comprising capturing patient insurance information for a patient associated with a medical service.

12. A method of claim 11, wherein the capturing of one or more of: the steps taken by a clinician in performing the medical service, and patient insurance information, includes extracting the data from the patient's electronic medical record (EMR).

13. A method of claim 9, further comprising maintaining records of activities and tasks associated with each patient.
